(19) United States
(12) Patent Application Publication
(10) Pub. No.: US 2021/0251553 A1
(43) Pub. Date: Aug. 19, 2021

(54) MULTIDIRECTIONAL BALLOON TIPPED CATHETER SYSTEM FOR CONDUCTING HIS BUNDLE SENSING AND PACING

(71) Applicant: EAST END MEDICAL LLC, Lewes, DE (US)

(72) Inventor: BRIJESHWAR S. MAINI, West Palm Beach, FL (US)

(21) Appl. No.: 17/173,513

(22) Filed: Feb. 11, 2021

Related U.S. Application Data
(60) Provisional application No. 62/977,973, filed on Feb. 18, 2020.

(51) Int. Cl. A61B 5/367; A61M 25/10; A61B 5/00; A61B 5/287; A61N 1/05

(52) U.S. Cl. CPC A61B 5/367; A61M 25/10; A61N 1/0573; A61B 5/287; A61B 5/6853

(57) ABSTRACT

The adoption of His Bundle Pacing (HBP) has been hindered by the procedural difficulty of achieving good lead position without causing cardiac damage or perforation. The procedural success rates are much lower when compared to traditional right ventricle (RV) pacing procedures due to the difficulty in mapping the His potential with the exposed helix and maintaining position while the pacing helix is fixated. The use of multidirectional balloon tipped catheter system that includes a catheter body with curls and flexion points and anchor balloon mounted to a distal end of the catheter body increases positional precision and an improved procedural success rate when compared to the conventional system.

### CROSS REFERENCE TO RELATED APPLICATIONS

This application claims the priority of U.S. Provisional Application Ser. No. 62/977,973, filed on Feb. 18, 2020, which is hereby incorporated herein by reference in its entirety.

### BACKGROUND

Natural Atrioventricular (AV) conducted ventricular contractions utilize the cardiac conduction system. This conduction system is a group of specialized cardiac muscle cells in the walls of the heart that send signals to the heart muscle causing it to contract. The main components of the cardiac conduction system are the sinoatrial (SA) node, AV node, bundle of His, bundle branches, and Purkinje fibers. The SA node (anatomical pacemaker) starts the sequence by causing the atrial muscles to contract. From there, the signal travels to the AV node, through the bundle of His, down the bundle branches (left and right bundles), and through the Purkinje fibers, causing the ventricles to contract. Patients who have AV nodal disease exhibit conduction issues between the atrial and ventricular chambers. Most often, this conduction issue occurs above bundle of His and presents as dyssynchrony of the atrial and ventricular chambers. If the conduction block occurs below the bundle of His and in only one of the left and right bundle branches (LBB and RBB), with intrinsic AV conduction occurring through the remaining intact bundles, but not both, the patient will exhibit a left or right Bundle Branch Block (LBBB or RBBB). Bundle branch blocks are identified by a delayed intrinsic ventricular electrical time. A complete bundle branch block (LBBB or RBBB) is identified as having an electrical activation time greater than 120 ms. Bundle branch blocks are not totally benign, particularly LBBB, and can lead to negative outcomes.

Currently, the standard pacing therapy for patients with AV conduction disease and requiring ventricular pacing, is to place a transvenous lead through the tricuspid valve and into the right ventricle (RV). This RV lead paces the ventricular myocardium which causes a cell by cell slow wave depolarization across the ventricles. This “cell by cell” depolarization of the ventricles utilizing a lead in the right ventricles causes the ventricles to contract in an unnatural way. With conventional RV pacing, the activation sequence of the ventricles is not the same as natural AV conducted contractions, with the right ventricle to depolarizing first and the left ventricle depolarizing slightly after. When patients are right ventricularly (RV) paced at a high percentage, there is an alarming progression of cardiac heart failure and pacing-induced cardiomyopathy. Pacing-induced cardiomyopathy (PICM) is most commonly thought of as a drop in left ventricular ejection fraction (LVEF) in the setting of chronic, high burden right ventricular (RV) pacing. It has been reported that about 20% of patients develop PICM after 3 to 4 years of RV pacing. These negative effects are thought to be a direct result of the unnatural contraction dynamics and resultant ventricular dyssynchrony resulting from RV pacing.

His Bundle Pacing (HBP) has emerged as an alternative to traditional RV pacing. By directly pacing the His bundle, HBP engages electrical activation of both ventricles by means of the cardiac Purkinje fiber network through the natural cardiac conduction system. This type of cardiac pacing may avoid ventricular dyssynchrony and preserve cardiac ejection fraction. Recent studies have also demonstrated the potential of HBP in correcting an underlying left bundle branch block and reversing cardiomyopathy caused by traditional RV pacing. HBP holds promise as an attractive mode to achieve physiological pacing. Widespread adaptation of this technique is dependent on enhancements in technology.

The His bundle lies in most people within the membranous portion of the interventricular septum, with a proportion of the proximal bundle lying on the right atrial portion of the septum, superior to the tricuspid valve annulus. The His bundle is surrounded by fibrous connective tissue rather than myocardium, and then enters the muscular septum and divides to form the right and left bundles. It has been demonstrated that transvenous HBP could reduce QRS duration and normalize electrocardiographic appearances in patients with bundle branch block. There is currently widespread consensus regarding the benefits of HBP and enthusiasm that this therapy may prove patient outcomes.

The current procedural method of achieving HBP involves employing a lead with a fixed screw helix for fixation and one of two catheters to achieve lead position. The fixed helix pacing lead is advanced past the distal end of the catheters while manual catheters manipulation and unipolar mapping utilizing the exposed helix is preformed to locate the His bundle potential signal. Care must be taken when mapping inside the heart with the exposed fixed helix. This helix is fully exposed and can cause local intracardiac surface edema while surface mapping which can mask the His bundle potential. The process of mapping with the exposed helix can often create an acute bundle branch block which may or may not resolve with time. Another pitfall to avoid is the potential for tissue to become stuck in the exposed lead helix which prevents adequate mapping and fixation of the lead helix. The target region for HBP is relatively robust and the current catheters are rather flimsy, which avoids the potential for catheter perforation, although the risk of perforation is possible if the catheter is advanced unintentionally into the incorrect position. The adoption of HBP has been hindered by the procedural difficulty of achieving good lead position without causing cardiac damage or perforation. The procedural success rates are much lower when compared to traditional RV pacing procedures due to the difficulty in mapping the His potential with the exposed helix and maintaining position while the pacing helix is fixated. The tools currently employed are currently simplistic and lacking the ability to adjust to varying anatomical differences or accurately maintain position within the beating heart during the implant procedure.

### SUMMARY

Embodiments of the disclosed invention provide a solution through a multidirectional balloon tipped catheter with sensing capability and will lead to increased procedural success and more widespread adoption of His Bundle Pacing (HBP) that has not been used prior and solves this problem.

These advantages and others are achieved, for example, by a multidirectional balloon tipped catheter system for conducting His bundle sensing and pacing. The catheter system includes a multidirectional catheter body having a proximal end and a distal end. The catheter body includes a plurality of curls and flexion points for multidirectional deflections. The catheter body further includes a plurality of lumens which include a pacing lead lumen including an exit port at the distal end and at least one balloon lumen including a balloon port near the distal end. The catheter system further includes an anchor balloon mounted to near the distal end of the catheter body, one or more mapping electrodes mounted to the distal end portion of the catheter body, and a pacing lead placed in the pacing lead lumen. The anchor balloon is in fluid communication with the balloon port and overhangs the distal end of the catheter body by a predetermined distance when the anchor balloon is inflated. The one or more mapping electrodes are configured to sense His bundle potential. The pacing lead is configured to protrude beyond the distal end of the catheter body when the pacing lead is in use.

The anchor balloon may be inflated with a fluid including air, saline, or contrast, and may be configured to be inflated in various sizes. The anchor balloon may be configured to expose the one or more mapping electrodes when the anchor balloon is deflated. The anchor balloon may overhang the distal end of the catheter body by two to three millimeters when the anchor balloon is inflated. The anchor balloon is a hydrophilic balloon.

The one or more mapping electrodes may include a first mapping electrode disposed at the distal end of the catheter body and a second mapping electrode disposed on the catheter body and spaced apart from the first mapping electrode. The first and second mapping electrodes may form a bipolar sensor. A diameter of the pacing lead lumen may be equal to or greater than 0.91 mm. A distance of a distal end of the anchor balloon from the distal end of the catheter body may be in a range of 10 mm to 20 mm when the anchor balloon is deflated. The pacing lead may include a screw helix. The catheter body may be configured to be insertable into a subclavian vein or other vascular access to approach His bundle. The plurality of lumens may further include one or more wiring lumens that house electrical wires connected to the one or more mapping electrodes.

These advantages and others are achieved, for example, by a method for conducting His bundle sensing and pacing with a multidirectional balloon tipped catheter system including a multidirectional catheter body. The method includes inserting the catheter system into a subclavian vein or vascular access, guiding the catheter system towards His bundle, sensing His bundle potential with one or more mapping electrodes disposed near the distal end of the catheter body, positioning a distal end of the catheter body at a location of the His bundle that is determined to be appropriate for pacing, anchoring the distal end of the catheter body at the appropriate location with inflated anchor balloon, and implanting a pacing lead into the appropriate location of the His bundle. The catheter system includes the anchor balloon mounted to the distal end portion of the catheter body, and the anchor balloon is inflated with a fluid supplied through at least one balloon lumen formed in the catheter body. The pacing lead is disposed in a pacing lead lumen formed in the catheter body and advances beyond the distal end of the catheter body while being implanted into the appropriate location of the His bundle.

### BRIEF DESCRIPTION OF THE DRAWINGS

The preferred embodiments described herein and illustrated by the drawings hereinafter be to illustrate and not to limit the invention, where like designations denote like elements.

FIGS. 1A-1C show an embodiment of multidirectional balloon tipped catheter system of the disclosed invention for sensing His bundle and positioning pacing lead.

FIGS. 2A-2C show side views of the distal end portion of the multidirectional balloon tipped catheter system.

FIG. 2D shows a cross-sectional view of the section A-A′ of the distal end portion of the multidirectional balloon tipped catheter system shown in FIG. 1C.

FIG. 3 shows an exemplary embodiment of a deflection mechanism that may be employed to control deflections of the distal end portion of the multidirectional balloon tipped catheter system.

FIG. 4, shows a workflow diagram for a method for conducting His bundle sensing and pacing with the multidirectional balloon tipped catheter system.

### DETAILED DESCRIPTION

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

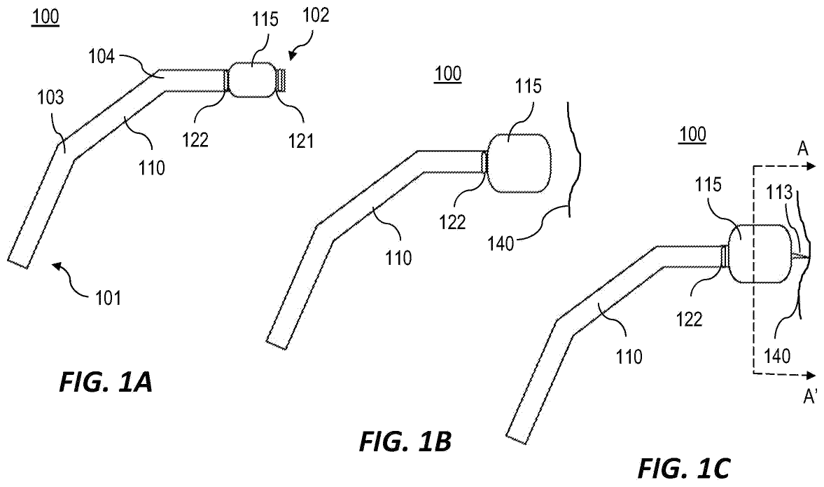

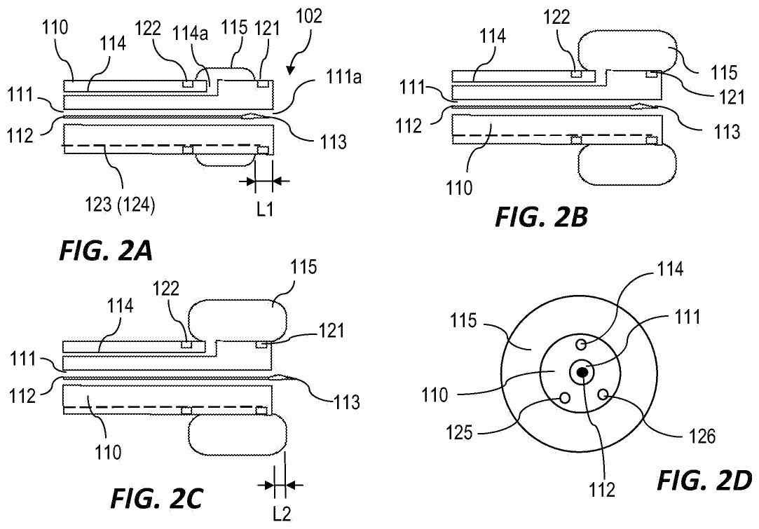

With reference to FIGS. 1A-1C shown are an embodiment of multidirectional balloon tipped catheter system 100 of the disclosed invention for sensing His bundle and positioning pacing lead. With reference to FIGS. 2A-2C shown are side views of the distal end portion of the multidirectional balloon tipped catheter system 100. With reference to FIG. 2D, shown is a cross-sectional view of the section A-A′ of the distal end portion of the multidirectional balloon tipped catheter system 100.

The multidirectional balloon tipped catheter system 100 includes a multidirectional or deflectable flexible catheter body 110 that includes a proximal end 101 and a distal end 102. The catheter body 110 is French sizes, and includes curls and flexion points to be multidirectional or deflectable. For example, the catheter body 110 may include a plurality of flexion points 103, 104 to facilitate the multidirectional deflections or bending. The catheter body 110 has a length sufficient to reach a selected location in a patient's cardiac structures. The catheter body 110 is configured to be insertable into a subclavian vein or other vascular access to approach His bundle. The catheter body 110 includes a plurality of lumens. The plurality of lumens include at least a wire lumen 111 for cord 112 connected to a pacing lead 113. The wire lumen 111 includes a wire access port (not shown) accessible to an operator at said catheter proximal 101 end and a wire exit port 111a at said flexible catheter distal end 102, and a balloon lumen 114 for inflating and deflating at least one anchor balloon 115. The balloon lumen 114 includes a balloon control port (not shown) for connecting to balloon control device accessible to an operator at the catheter proximal end 101 and a balloon port 114a near the multidirectional catheter distal end 102. The plurality of lumens may further include other lumens such as lumens 125, 126 for wires 123, 124 connected to mapping electrodes 121, 122.

The multidirectional balloon tipped catheter system 100 includes compliant or non-compliant anchor balloon 115 that is mounted on the multidirectional catheter body 110 near the distal end 102 of the catheter body 110. FIGS. 1A and 2A show deflated anchor balloon 115, and FIGS. 1B and 2B show inflated anchor balloon 115 at the distal end 102 portion of catheter body 110. The anchor balloon 115 is connected to the balloon port 114a of the catheter lumen 114, and is in fluid communication through the balloon lumen 114. Fluid, which is injected or removed at the balloon control port at the proximal end 101, inflates or deflates the anchor balloon 115 through the balloon lumen 114.

The anchor balloon 115 may be inflated with air, saline, contrast and other solutions, and may be inflated to various sizes. The anchor balloon 115 is placed at a selected distance from the distal end 102 of the catheter body 110. For example, when the anchor balloon 115 is deflated, the distance L1 of a distal end of the anchor balloon 115 from a distal end 102 of the catheter body 110 may be in the range 10 mm (0.4 inches) to 20 mm (0.8 inches). When the anchor balloon 115 is inflated, the anchor balloon 115 may overhang the distal end 102 of the catheter body 110 by a distance L2 which may be two to three millimeters.

The multidirectional balloon tipped catheter system 100 includes at least one mapping electrode 121 near the distal end 102 of the catheter body 110. The mapping electrode 121 allows atraumatic mapping of the His bundle potential. The mapping electrode 121 works as a unipolar sensor for detecting and mapping the His bundle potential. In another embodiment, the catheter system 100 may include second mapping electrode 122 that is placed a few millimeters behind the first mapping electrode 121 (toward proximal end 101). In this configuration, mapping electrodes 121, 122 together work as a bipolar sensor for atraumatic mapping of the His bundle potential, making bipolar sensing possible. The anchor balloon 115 is configured such that the mapping electrode 121 is exposed at the distal end 102 of the catheter body 110 when the anchor balloon 115 is deflated so that mapping the His bundle potential may be performed by using the electrode 121 and/or electrodes 121, 122 to find an appropriate location of the heart tissue 140 for His bundle pacing.

The catheter body 110 may include wiring lumens 125, 126 that house electrical wires 123, 124 connected to the mapping electrodes 121, 122. The wires 123, 124 at the proximal end 101 of the catheter body 110 may be coupled to an external device that may send signals to or receive signals from the mapping electrodes 121, 122.

The multidirectional balloon tipped catheter system 100 includes pacing lead 113 that is connected to cord 112 disposed in the lumen 111. The catheter body 110 includes pacing lead lumen 111 that includes a cord access port (not shown) accessible to an operating device at the proximal end 101 and exit port 111a at the distal end 102 of the catheter body 110. The pacing lead lumen 111 may be positioned at a center of the cross-section of the catheter 110 as shown in FIG. 2D. The diameter of the pacing lead lumen 111 may be equal to or greater than 0.91 mm (0.035 inches). The pacing lead 113 may be placed inside the pacing lead lumen 111 while mapping His bundle potential and positioning the catheter system 100 against heart tissue 140. The pacing lead 113 may advance out of the pacing lead lumen 111 beyond the distal end 102 of the catheter body 110 to be placed or implanted on the heart tissue 140. The pacing lead 113 may have a form of screw helix. FIGS. 1C and 2C show the pacing lead 113 advancing out of the distal end 102 of the catheter body 110.

The anchor balloon 115 may be used in an atraumatic fashion over cardiac structures. The anchor balloon 115 may glide over the cardiac structures, while sensing is performed by using the mapping electrodes 121, 122 to obtain the best site for lead implantation. Once an appropriate location of the heart tissue 140 is determined for His bundle pacing, the catheter system 100 is used as a conduit for implantation of the pacing lead 113. The anchor balloon 115 may be inflated to anchor the distal end 102 of the catheter body 110 at the appropriate location of the heart tissue 140. When the distal end 102 of the catheter body 110 with the inflated anchor balloon 115 is positioned and stabilized at the location, the pacing lead 113 may be advanced to be implanted in the heart tissue 140. Once the pacing lead 113 is in place, the catheter body 110 may be removed using multiple methods which may include slitting and splitting the catheter body, or may be removed by methods that are general practices for lead implantation.

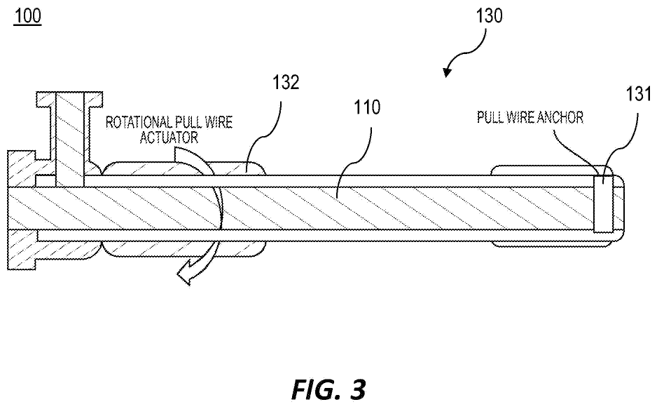

With reference now to FIG. 3, shown is an exemplary embodiment of a mechanical deflection device 130 that can be employed at the proximal end 101 portion of the catheter body 110 to control deflections of the distal end portion of the catheter system 100. Mechanical deflection mechanism may enable distal end of catheter body 110 to be deflected or angulated to various angles with respect to a longitudinal axis (from the proximal end 101 to the distal end 102) of the catheter system 100. Mechanical deflection device 130 may include a pull wire anchor 131 affixed to the catheter body 110 and pull wire actuator 132 connected to pull wire anchor 131 with pull wire (not shown). Rotation of pull wire actuator 132, as shown, may exert force on pull wire anchor 131 that deflects or angulates distal end of the catheter body 110. Pull wire actuator 132 may be rotated by handle connected thereto (not shown). The deflection device 130 together with the flexion points and curls formed in the catheter body 110 enables the catheter body 110 to easily navigate over heart structures. U.S. patent application Ser. No. 17/061,761 filed on Oct. 2, 2020 by the same inventor discloses an improved handle that can be used for the catheter system 100 of the disclosed invention to provide deflections.

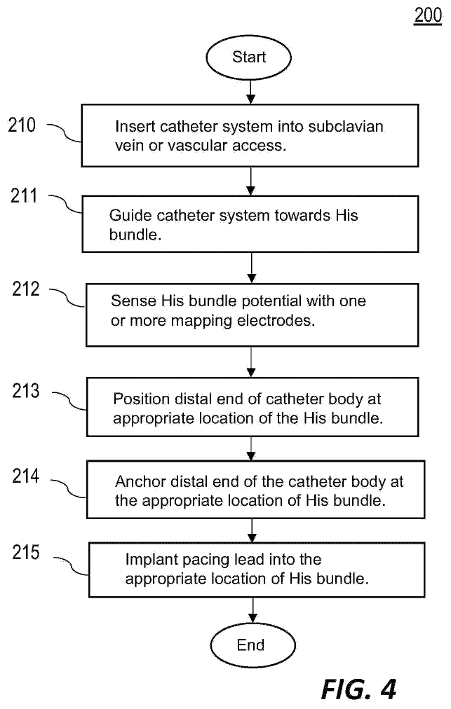

With reference to FIG. 4, shown is a workflow diagram for a method 200 for conducting His bundle sensing and pacing with a multidirectional balloon tipped catheter system 100 including a multidirectional catheter body 110. The catheter system 100 is inserted into a subclavian vein or vascular access, block 210. The catheter system 100 is guided towards His bundle, block 211. The catheter system 100 senses His bundle potential with one or more mapping electrodes 121, 122 disposed at a distal end portion of the catheter body, block 212. The distal end of the catheter body 110 is positioned at a location of the His bundle 140 that is determined to be appropriate for pacing, block 213. The distal end of the catheter body 110 is anchored at the appropriate location with inflated anchor balloon 115, block 214. A pacing lead 113 is implanted into the appropriate location of the His bundle, block 215. After the pacing lead 113 is implanted, the catheter body 110 with deflated anchor balloon 115 may be removed while leaving the pacing lead 130 in place.

The anchor balloon 115 is atraumatic and allows for use of a more robust catheter designs. The increased rigidity of the catheter body facilitates increased positional precision and an improved procedural success rate. In an embodiment, the anchor balloon 115 may be a hydrophilic balloon with a surface having hydrophilic nature. The catheter system 100 of the disclosed invention provides advantages over the conventional devices. Unlike the conventional devices, the pacing lead 113 of the catheter system 100 of the disclosed invention is not exposed while the catheter system 100 maps the His bundle potential to find an appropriate location of heart tissues for His bundle pacing, preventing any issues that can be caused by exposed screw helix in the conventional devices. The catheter system 100 of the disclosed invention utilizes atraumatic anchor balloon that allows maneuvering of the catheter system 100 over cardiac structures without causing any injuries or damages to heart tissues and also allows to use more rigid multidirectional catheter body, which increases the ability to adjust to varying anatomical differences and to accurately maintain position within the beating heart during the implant procedure.

Since many modifications, variations, and changes in detail can be made to the described preferred embodiments of the invention, it is intended that all matters in the foregoing description and shown in the accompanying drawings be interpreted as illustrative and not in a limiting sense. Consequently, the scope of the invention should be determined by the appended claims and their legal equivalents.

What is claimed is:

1. A multidirectional balloon tipped catheter system for conducting His bundle sensing and pacing, comprising:
a multidirectional catheter body having a proximal end and a distal end and including a plurality of curls and flexion points for multidirectional deflections, wherein the catheter body includes a plurality of lumens comprising:
a pacing lead lumen including an exit port at the distal end; and
at least one balloon lumen including a balloon port near the distal end;
an anchor balloon mounted to near the distal end of the catheter body, wherein the anchor balloon is in fluid communication with the balloon port and wherein the anchor balloon overhangs the distal end of the catheter body by a predetermined distance when the anchor balloon is inflated;
one or more mapping electrodes mounted to the distal end portion of the catheter body, wherein the one or more mapping electrodes are configured to sense His bundle potential; and
a pacing lead placed in the pacing lead lumen, wherein the pacing lead is configured to protrude beyond the distal end of the catheter body when the pacing lead is in use.

2. The multidirectional balloon tipped catheter system of claim 1 wherein the anchor balloon is inflated with a fluid including air, saline, or contrast.

3. The multidirectional balloon tipped catheter system of claim 1 wherein the anchor balloon is configured to be inflated in various sizes.

4. The multidirectional balloon tipped catheter system of claim 1 wherein the anchor balloon is configured to expose the one or more mapping electrodes when the anchor balloon is deflated.

5. The multidirectional balloon tipped catheter system of claim 1 wherein the anchor balloon overhangs the distal end of the catheter body by two to three millimeters when the anchor balloon is inflated

6. The multidirectional balloon tipped catheter system of claim 1 wherein the one or more mapping electrodes comprises:
a first mapping electrode disposed at the distal end of the catheter body; and
a second mapping electrode disposed on the catheter body and spaced apart from the first mapping electrode, wherein the first and second mapping electrodes form a bipolar sensor.

7. The multidirectional balloon tipped catheter system of claim 1 wherein a diameter of the pacing lead lumen is equal to or greater than 0.91 mm.

8. The multidirectional balloon tipped catheter system of claim 1 wherein a distance of a distal end of the anchor balloon from the distal end of the catheter body is in a range of 10 mm to 20 mm when the anchor balloon is deflated.

9. The multidirectional balloon tipped catheter system of claim 1 wherein the pacing lead includes a screw helix.

10. The multidirectional balloon tipped catheter system of claim 1 wherein the anchor balloon is a hydrophilic balloon.

11. The multidirectional balloon tipped catheter system of claim 1 wherein the catheter body is configured to be insertable into a subclavian vein or other vascular access to approach His bundle.

12. The multidirectional balloon tipped catheter system of claim 1 wherein the plurality of lumens further comprises one or more wiring lumens that house electrical wires connected to the one or more mapping electrodes.

13. A method for conducting His bundle sensing and pacing with a multidirectional balloon tipped catheter system comprising a multidirectional catheter body, comprising:
inserting the catheter system into a subclavian vein or vascular access;
guiding the catheter system towards His bundle;
sensing His bundle potential with one or more mapping electrodes disposed near the distal end of the catheter body;
positioning a distal end of the catheter body at a location of the His bundle that is determined to be appropriate for pacing;
anchoring the distal end of the catheter body at the appropriate location with inflated anchor balloon, wherein the catheter system includes the anchor balloon mounted to the distal end portion of the catheter body, and wherein the anchor balloon is inflated with a fluid supplied through at least one balloon lumen formed in the catheter body; and
implanting a pacing lead into the appropriate location of the His bundle, wherein the pacing lead is disposed in a pacing lead lumen formed in the catheter body and advances beyond the distal end of the catheter body while being implanted into the appropriate location of the His bundle.

14. The method of claim 13 further comprising removing the catheter body while the pacing lead is implanted in the His bundle.

15. The method of claim 13 wherein the anchor balloon is inflated with a fluid including air, saline, or contrast.

16. The method of claim 13 wherein the anchor balloon is configured to expose the one or more mapping electrodes when the anchor balloon is deflated.

17. The method of claim 13 wherein the anchor balloon overhangs the distal end of the catheter body by two to three millimeters when the anchor balloon is inflated

18. The method of claim 13 wherein the one or more mapping electrodes comprises:
a first mapping electrode disposed at the distal end of the catheter body; and
a second mapping electrode disposed on the catheter body and spaced apart from the first mapping electrode, wherein the first and second mapping electrodes form a bipolar sensor.

19. The method of claim 13 wherein the pacing lead includes a screw helix.

20. The method of claim 13 wherein the anchor balloon is a hydrophilic balloon.

21. The method of claim 13 wherein the catheter body include a plurality of curls and flexion points for multidirectional deflections.
